(19) United States
(12) Patent Application Publication
(10) Pub. No.: US 2005/0015045 A1
(43) Pub. Date: Jan. 20, 2005

(54) Manual breast pump

(76) Inventors: Mitsuo Tashiro, Tokyo (JP); Hiroyuki Uehara, Tokyo (JP); Yuko Odabayashi, Tokyo (JP)

Correspondence Address: CERMAK & KENEALY, LLP, 23 W. Myrtle St, Alexandria, VA, 22301, US

(21) Appl. No.: 10/884,197

(22) Filed: Jul. 2, 2004

Related U.S. Application Data
(63) Continuation of application No. PCT/JP03/01223, filed on Feb. 6, 2003.

(30) Foreign Application Priority Data
Aug. 29, 2002 (JP) 2002-251463

(51) Int. Cl. A61M 1/06

(52) U.S. Cl. 604/74

(57) ABSTRACT

A manual breast pump which can be handled conveniently and disassembled with ease for cleaning or other purposes although it would not be disassembled by error in operation. The manual breast pump can include a conical suction portion, which is made to abut a breast and includes a breast pump main body that is held in communication with a bottle, and a lever with long shape in a direction and detachably fitted to the breast pump main body at a middle position thereof by means of a rotative and supportive shaft. The lever is adapted to turn in opposite directions around the shaft. The lever is operated so as to relatively reciprocate a piston and generate negative pressure so that the suction portion that is held in communication with an internal space of the lever sucks milk and causes the sucked milk to fall into the bottle.

This application is a continuation of and claims priority under 35 U.S.C. §120 to PCT/JP2003/001223 filed on Feb. 6, 2003 in Japan designating the U.S., and claims priority under 35 U.S.C. §119 to JP2002-251463 filed on Aug. 29, 2003 to which PCT/JP2003/001223 claims priority, both of which are hereby incorporated by reference.

### 1. FIELD OF THE INVENTION

This invention relates to an improvement of a manual breast pump, for example.

### BACKGROUND OF THE INVENTION

As a manual breast pump, there is a one that was proposed by the applicant of the present patent application and illustrated in FIG. 13 of the accompanying drawings (Jpn. Pat. Appln. Laid-Open Publication Hei. 1-190364).

The manual breast pump 1 comprises a breast pump main body 5 that is provided with a suction portion 2 and can be detachably fitted to a bottle 6. A vent pipe 3, which may typically be a rubber pipe, extends from the breast pump main body 5. And a negative pressure generating means 4, which is a hollow rubber member, is fitted to the opposite end of the vent pipe 3.

The user holds the bottle 6 with one of his or her hands and fit the horn-shaped suction portion 2 to a breast at the opening thereof. As the user repeatedly grasp the negating means pressure generating means 4 with the other hand to generate negative pressure, which is transmitted to the suction portion 2 by way of the vent pipe 3 and the breast pump main body 5, mother's milk is sucked and falls into the bottle 2.

Such a manual breast pump 1 is difficult to use because it needs to be operated by the two hands. Additionally, although the bottle 2 and the vent pipe 3 can be removed from the breast pump main body 5, the user can dismantle neither the breast pump main body 5 that has a number of branch passages nor the negative pressure generating means 4. So, it is difficult for the user to routinely clean the inside of the breast pump main body 5.

Thus, while the manual breast pump 1 needs to be held in a hygienically clean condition because it deals with mother's milk to be ingested by infants, it requires cumbersome maintenance operations.

In view of the above identified circumstances, one of several aspects of the present invention is to provide a manual breast pump that can be easily dismantled for cleaning and other purposes, cleaned with ease and handled in a hygienically clean condition.

### SUMMARY OF THE INVENTION

According to claim 1 of the present invention, an aspect of the invention can be achieved by providing a manual breast pump comprising:
a breast pump main body having a substantially conical suction portion to be fitted to a breast and an attach and detach portion detachably fitted to a bottle so as to communicate with the bottle and
a lever with long shape extending in a direction and detachably held by spindle on the breast pump main body at a middle part thereof by means of a spindle holding means, being held rotatably in opposite directions around the spindle
and the lever configured to suck milk from the suction portion communicating with a predetermined internal space that contains a piston by generating negative pressure in the internal space due to change of specified internal space that contains a piston by means of a relative reciprocating motion of the piston that is caused by a movement of an end of the lever as a result of a rotary motion thereof and make the sucked mother's milk drop into the bottle and a negative pressure generating means comprising a vent passage communicating with the suction portion, a fixed piston arranged at an end of the vent passage, a sleeve arranged integrally at an end of the lever, into which the fixed piston of the breast pump main body is inserted so as to communicate with the vent passage, the inserted fixed piston being adapted to slide in the inside of the sleeve when the lever is rotated so as to generate negative pressure by rotation of the lever in the space defined by the inner surface of the sleeve and a facet of the fixed piston.

With the configuration of claim 1 of the present invention the breast pump main body can be detachably fitted to a bottle by means of an attachable/detachable portion.

The breast pump is configured to generate negative pressure by operating the lever. More specifically, negative pressure is generated either by driving the piston by means of operation of the lever or by moving the space containing the piston. In short, negative pressure is generated by causing the piston to relatively reciprocate. Additionally, unlike conventional breast pump, the milker does not involve a long vent pipe, which is cumbersome and difficult to handle for maintenance or other purpose. The lever is removed from the breast pump main body so that the lever and the breast pump main body can be cleaned separately.

Furthermore, since the sleeve is integrally arranged at an end of the lever, the opening through which the fixed piston is inserted comes to be exposed when the lever is removed so that the sleeve can be cleaned with ease. In short, the space for generating negative pressure has a simple configuration and can be cleaned with ease. Additionally, the fixed piston without the sleeve becomes exposed at the breast pump main body only by removing the lever so that it can also be cleaned with ease. In other words, the mechanism for generating negative pressure becomes exposed so as to be ready for cleaning only simply when the lever is removed from the breast pump main body can be provided.

Thus, according to the invention, the manual breast pump disassembled with ease for cleaning and held in a hygienically clean state.

The breast pump as recited in claim 1, claim 2 of the invention is further comprising: the spindle holding means provided a bearing portion arranged at the side of the lever and a bearing portion arranged at the side of the breast pump main body; the spindle held by shaft member inserted into the spindle holes penetrated by each bearing portion laid one on the other; and the spindle member provided with an engaging means for engagement with any of the bearing sections.

With the configuration of claim 2 of the invention, the lever is held by spindle to the breast pump main body by the spindle member inserted into the spindle holes so that the breast pump can be assembled with ease and disassembled only by pulling the spindle member out of the spindle holes. In short, the breast pump can be assembled and disassembled with ease. Additionally, once the spindle member is inserted into the spindle holes, the lever can no longer be removed from the breast pump main body. Thus, while the lever can be easily fitted to and removed from the breast pump main body, it is effectually prevented from inadvertently coming off from the breast pump main body. Furthermore, once the spindle member is surely engaged in position, the lever is also effectually prevented from inadvertently coming off from the breast pump main body.

The breast pump as recited in claim 1 or 2, claim 3 of the invention is further comprising: at least the inner wall of the sleeve has a curved profile that agrees with the locus of the rotary motion of an end of the lever.

With configuration of claim 3 of the invention, lever can be turned easily and smoothly because its profile agrees with the locus of the rotary motion of an end of the lever.

The breast pump as recited in claim 1, 2 or 3, claim 4 of the invention is further comprising: the outer periphery of the piston to be elastic and to be inclined toward the lever to reduce its dimensions.

With the configuration of claim 4 of the invention, the piston and the sleeve can be airtightly put together to reliably generate negative pressure in the inside because the outer periphery of the piston is elastic. Additionally, the piston contacts the sleeve at the outermost periphery thereof due to the inclination so that the lever can be operated easily.

The breast pump recited in claim 1, 2, 3 or 4, claim 5 of the invention is further comprising: The piston no longer airtightly be contacted to the inner wall of the sleeve at the end of the sleeve. When the other end of the lever be moved to come closer to the bottle.

With the configuration of claim 5 of the invention, once the other end of the lever is made to come closer to the bottle to increase the negative pressure, air is allowed to leak into the inside so that the negative pressure is prevented from increasing excessively each time the lever is operated. Thus, the resistance against the operation of the lever can be prevented from becoming too large and can be prevented from an obstruction of may be operated smooth operation. At the same time, the negative pressure is made to fall quickly without difficulty. In other words, the negative pressure is made to change pulsatively.

The breast pump as recited in claim 1, 2, 3, 4 or 5, claim 6 of the invention is further comprising: the sleeve provided on the outer surface of its closed end with a pressure regulating means.

With the configuration of claim 6 of the invention, the pressure regulating means is exposed to the outside at a position where it can be operated with ease for the convenience of the operator. Additionally, the pressure regulating means is located at a position that allows it to be removed and cleaned with ease.

The breast pump recited in claim 6, claim 7 of the invention is further comprising: the pressure regulating means arranged on the outer surface of the closed end of the sleeve; a valve box having at least two pairs of oppositely disposed inner walls, the distance L1 separating one of the pairs of oppositely disposed inner walls being greater than the distance L2 separating the other pair of oppositely disposed inner walls; a valve made of an elastic material and having a substantially circular outer periphery and a slit for allowing air to pass through; and the opening of the slit being adjusted by inserting the valve in the valve box and rotating the valve in the valve box.

With the configuration of claim 7 of the invention, the pressure regulating means can be operated in a simple manner of rotating or turning the valve in the valve box. Additionally, the pressure regulating means is formed by using a minimal number of components including a valve box arranged on the outer surface of the sleeve and a valve so that it can be disassembled and cleaned with ease simply by taking out the valve out of the valve box.

### BRIEF EXPLANATION OF THE DRAWINGS

FIG. 1 is a schematic perspective view of the first embodiment of breast pump according to the invention.

FIG. 2 is a schematic cross sectional view of the embodiment of breast pump of FIG. 1.

FIG. 3 is a schematic partial cross sectional view of the embodiment of breast pump of FIG. 1, illustrating the structure of the spindle holding for securing the lever to the breast pump.

FIG. 4 is a schematic illustration of the embodiment of breast pump of FIG. 1, illustrating the structure of the engagement the spindle holding.

FIG. 5 is a schematic lateral view of the embodiment of breast pump of FIG. 1.

FIG. 6 is a schematic cross sectional view of the embodiment of breast pump of FIG. 1 when the embodiment is operated for sucking milk.

FIG. 7 is a schematic cross sectional view of the embodiment of breast pump of FIG. 1 when the embodiment is operated for leaking air.

FIG. 8 is a schematic perspective view of the valve main body of the pressure regulating means arranged on the outer surface of the sleeve of the embodiment of breast pump of FIG. 1, the valve main body being part of the valve structure.

FIG. 9 is a schematic perspective view of the valve box that is combined with the valve main body, which is part of the valve structure, to form the pressure regulating means that is arranged on the outer surface of the sleeve of the embodiment of breast pump of FIG. 1.

FIG. 10 is a schematic illustration of operation of the pressure regulating means that is arranged on the outer surface of the sleeve of the embodiment of breast pump of FIG. 1.

FIG. 11 is another schematic illustration of operation of the pressure regulating means that is arranged on the outer surface of the sleeve of the embodiment of breast pump of FIG. 1.

FIG. 12 is still another schematic illustration of operation of the pressure regulating means that is arranged on the outer surface of the sleeve of the embodiment of breast pump of FIG. 1.

FIG. 13 is a schematic illustration of a conventional breast pump.

### DETAILED DESCRIPTION OF PREFERRED EMBODIMENTS

Now, preferred embodiments of the present invention will be described in detail by referring to the accompanying drawings.

The following embodiments include various preferable technical limitations because they are preferred concrete embodiments of invention. However, the scope of the present invention is not limited to them.

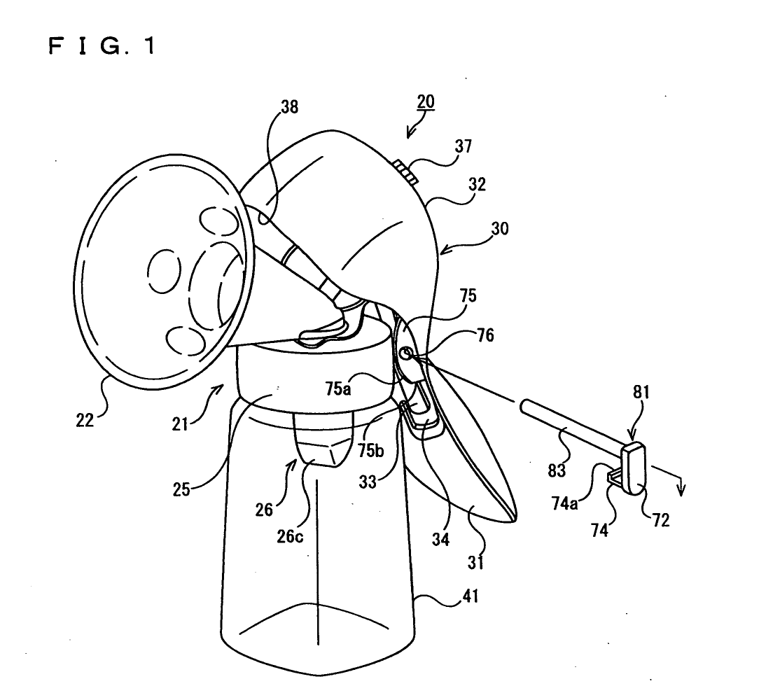

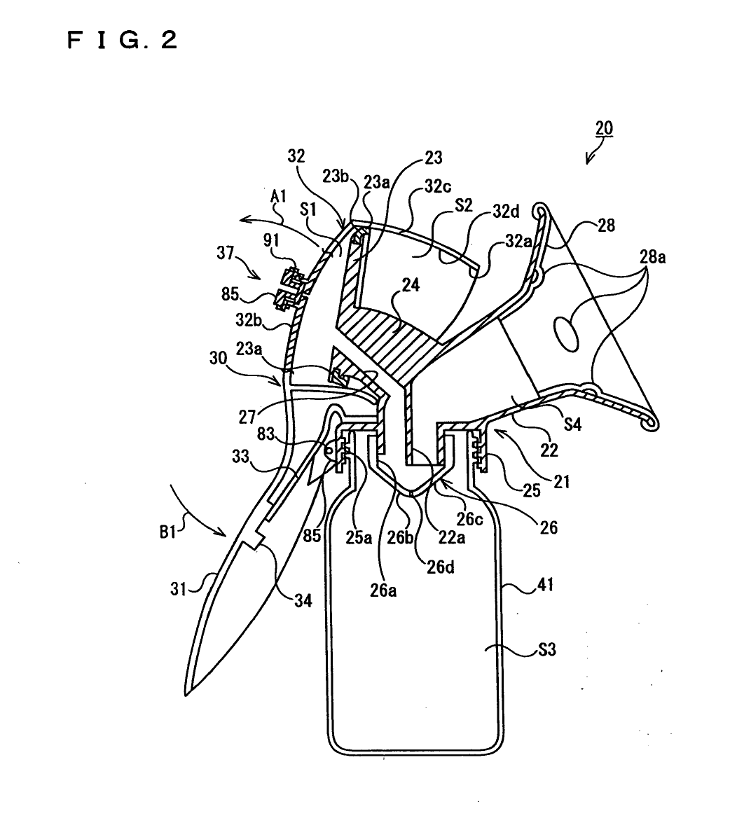

FIG. 1 is a schematic perspective view of an embodiment of breast pump 20 according to the invention, illustrating its overall configuration. FIG. 2 is a schematic cross sectional view of the embodiment. As shown in FIGS. 1 and 2, the breast pump 20 comprises a breast pump main body 21 (to be referred to simply as “main body” hereinafter) and a lever 30 (to be also referred to as “handle”), of which the lever 30 can be attach and detach to the breast pump main body 21.

The main body 21 is made of a synthetic resin material that is relatively light, tough and easily moldable. Synthetic resin materials that can be used for the main body 21 include polycarbonate, polycycloolefin and polyethersulfone and the like. The main body 21 comprises an attach and detach portion 25 to attach and detach to a bottle 41 for storing sucked milk. As shown in FIG. 2, the attached detach portion 25 has a flat and cylindrical profile and is typically provided with an internal thread 25a so that it may be engaged with the external thread formed around the neck of the bottle 41. The bottle 41 may be one that is dedicated to the breast pump 20 or any other one that can be engaged with the attach and detach portion 25.

A conical or horn-shaped suction portion 22 inclined with its opening facing the outside are arranged at the upper portion of the attach and detach portion 25 of the main body 21. A buffer 28 made of an elastic material such as silicon, elastomer or natural rubber is detachably fitted to the suction portion 22 at a position close to the open end of the suction portion. The buffer 28 portion reduces the stimulus given to the breast touched by suction portion 22 during the milking operation so that the breast may be free from pain. The buffer 28 portion has on the inner peripheral surface thereof a number of projections 28a. The suction portion 22 has vent passage 22a, which allows both air and sucked milk to pass through it and is bent downward and directed toward the bottle 41. The opening and its periphery of the suction portion vent passage 22a of the suction portion 22 are located inside the main body 21 and the attach and detach portion, fit with a cellular valve 26. Another vent passage 27 is provided adjacent to the suction portion vent passage 22a. As shown in FIG. 2, the bottom end opening of the vent passage 27 is communicating with the suction portion vent passage 22a within the cell 26a of the cellular valve 26 and the vent passage 27 runs upward through piston rod 24 and terminates at the top end opening. The lower end of the suction portion vent passage is lengthened than the lower end of the vent passage 27 so that sucked milk can hardly enter the vent passage 27.

As seen in FIGS. 1 and 2, the cellular valve 26 has a cap-like profile as a whole and is made of an elastic material such as silicon, elastomer or natural rubber. The oppositely disposed elastic lateral walls 26b, 26c of the cellular valve 26 are inclined so as to come closer to each other as they approach the lower ends thereof. Slit 26d is constructed at the bottom end where both wall 26b and 26c come closely. Thus, when the volume of sucked milk stored in the cell 26a gets to a predetermined amount, the weight on the change of the negative pressure when negative pressure released as described later make leading edge of the lateral walls 26b, 26c open to allow milk to fall into the bottle 41. Due to the slit 26d constructed at the lower ends of the inclined lateral walls, the cell 26a is preventing from air coming into from the bottle 41 when negative pressure prevails in the inside.

The main body 21 has a fixed piston 23 that is arranged at the top of and integral with the above described piston rod 24. While both the piston and the piston rod are stationary in this embodiment, the piston may be movable. The fixed piston 23 is arranged transversally at the top of the piston rod 24 in a direction opposite to the direction in which the suction portion 22 extends. The fixed piston 23 has a disk-shaped or an elliptic profile.

In FIG. 2, the lever 30 has a shape prolonged approximately upward and downward and is provided at an end thereof, or the top end in FIG. 2, with a sleeve 32, which is integral with the lever 30 and has an opening facing the above described fixed piston 23. The lever 30 is a molded product and as a whole made of relatively lightweight synthetic resin such as polycarbonate, polycycloolefin or polyethersulfone.

The other end of the lever 30 has a handle-like profile which extends downward as shown in FIG. 2 to form a lever main body 31 as operating part.

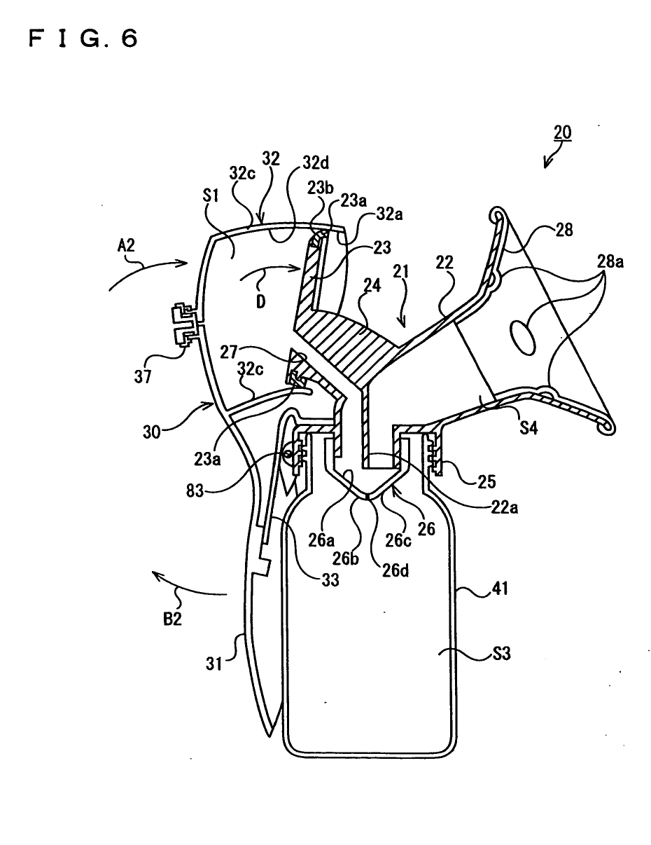

The lever 30 can be attached and detached to the main body 21 in a manner as will be described hereinafter. When the lever 30 is fixed to its position, it is secured to the main body 21 at a longitudinal middle position thereof by means of a spindle holding so that it may be rotate or turned or swinged around the spindle in two opposite directions, or the directions of arrows A1 and B 1 in FIG. 2 or arrows A2 and B2 in FIG. 6.

An oblong leaf spring 33 or the like is arranged as urging means in the inside of the lever 30. The leaf spring 33 is bent along an edge thereof and the bent section of the leaf spring 33 is secured in position as it is pinched by a holding means 21b fitted to the main body 21, while the opposite edge of the leaf spring 33 is secured in position as it is made to abut a spring bearing 34 of which other end arranged near the front end of the lever main body 31 of the lever 30. With this arrangement, the lever main body 31 of the lever 30 is urged by the leaf spring 33 in the direction of arrow B2.

The sleeve 32 integrally arranged at an end of the lever 30, or at the top end in FIG. 2, has a closed end portion of which end edge portion 32b is closed. And location of the fixed piston 23 is an opening 32a. The wall portion 32c surrounding the opening 32a defines cylinder-shaped internal spaces S1, S2 that are open only at an end. While the internal spaces S1, S2 is a single space, the single space is divided into an open space S2 that is open to the atmosphere and a closed space S1 that is defined by the (inner) wall section 32c and the inside of the end section 32b of the sleeve 32 when the fixed piston 23 is inserted into the sleeve 32 through the opening 32a.

A pressure regulating means 37 is fitted to the outer surface of the closed end section 32b which is the closed end of the sleeve 32 so as to make the pressure of the internal space S1 adjustable. A number of projections may be arranged on the inner wall of the closed end section 32b of the sleeve 32 to such a height that they may not hit the piston 23 for the purpose of reducing the volume of air in the internal space S1 and boosting the negative pressure in the inside when the breast pump is in operation. At least the inner wall 32d of the sleeve 32 is made to show a profile that substantially agree with the locus of the rotary motion of the top end and that of the bottom end of the lever 30. Thus, the sleeve 32 shows a profile that substantially agree with the locus of the rotary motion of the top end and that of the bottom end of the lever 30, the lever 30 can be moved to rotate smoothly and easily.

On the other hand, the fixed piston 23 arranged at the side of the main body 21 has a profile that allows the piston to tightly contact the inner wall of the sleeve 32 and a ring-shaped packing 23a that is an elastic member and operates as piston ring is fitted to the flange-shaped end of the fixed piston. At least a part 23b of the outer periphery of the packing 23a is inclined toward the lever 30 (left side in FIG. 2) so as to reduce its dimensions. Due to the inclination, as the outermost periphery of the elastic packing 23a abuts the entire periphery of the inner wall 23d, the airtight closure is maintained and the lever 30 can be operated lightly.

As shown in FIG. 1, the wall of the sleeve 32 of the lever 30 is partly indented at a position near the top end of the sleeve 32 to produce a leak section 38. This section is located at the end of the sleeve 32 in terms of the relative reciprocating motion of the fixed piston 23, which will be described hereinafter. This portion functions in a state when negative pressure is maximized.

Thus, for an operation of breaking negative pressure, being partly opened to the atmosphere by way of the leak section 38 by only the fixed piston 23 being moved to the leak section 38 so that the lever 30 can be operated easily without exerting extremely large force. It may be so arranged that the entire peripheral edge of the fixed piston 23 or an upper region thereof simultaneously comes out of the sleeve 32 for an operation of breaking negative pressure.

As will be described hereinafter, any increase of negative pressure can be rapidly reduced to smoothly and pulsatively change negative pressure by allowing air to leak after the lever main body 31 is moved toward the bottle 41 to consequently increase negative pressure.

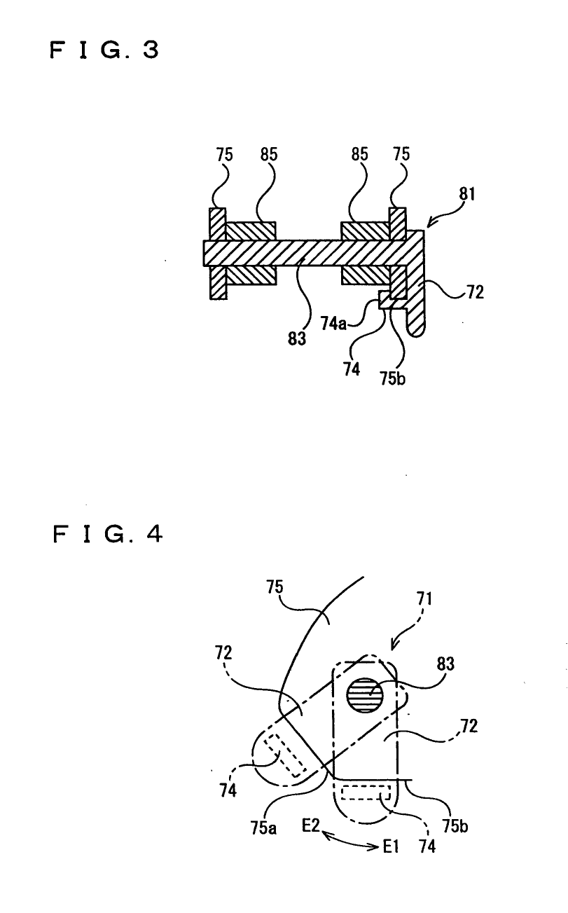

Now, the arrangement of securing the lever 30 to the main body 21 by means of a spindle holding will be described by referring to FIGS. 3 and 4.

FIG. 3 shows an example of a structure that can be used to rigidly secure the lever 30 of the breast pump 20 to the attach and detach portion 25.

The attach and detach portion 25 is provided as integral portion thereof with a pair of bearing portions 85, 85 for the main body 21. On the other hand, the lever 30 is provided with a pair of bearing portions 75, 75 that are located outside of the width between bearing portions 85 and 85.

An oblong spindle member 81 is provided with a base portion 72 and a projection or ridge 74. The spindle member 81 has a spindle 83 that is inserted in such a way it penetrate the lever side bearings 75, 75 and the attach and detach portion side bearings 85, 85. A holding pawl 74a is arranged at the front end of the projection or ridge 74 in order to prevent the spindle member 81 from inadvertently coming off from the bearing portion.

As shown in FIG. 4, the bearing portion 75 of the lever 30 is provided with a sloped surface 75a and a horizontal engaging surface 75b continued to the sloped surface 75a at a lower portion of the bearing portion 75.

Then, the bearing portion 75 of the lever 30 is placed on the corresponding bearing portion (not shown) of the main body 21 and the spindle 83 of the spindle member 81 is driven to run through the spindle holes 76, 76 of the bearing portions 75 from the both outside of each bearing portion 75.

As showing in FIG. 4, firstly, the spindle member 81 is inclined. Then, as the spindle 83 is driven to run through the spindle holes 76 and the spindle member 81 is rotated around the spindle 83 in the direction of arrow E1, the projection 74 comes to be engaged with or held by the horizontal engaging surface 75b.

As a result, the lever 30 is secured to the main body 21.

Conversely, to remove the lever 30 from the main body 21, the spindle member 81 is rotated in the direction of arrow E2 in FIG. 4. Then, the projection 74 is released from the horizontal engaging surface 75b. As a result, the lever 30 can be removed from the main body 21.

In this way, the lever 30 is securely held only by the spindle member 81 be inserted into the spindle holes 76 and by the operation for mutual engagement. Therefore, the assembling operation is very simple and easy. Additionally, the lever 30 can be removed from the main body 21 simply by removing the spindle member 81 from the spindle holes 76 so that assembling and disassembling is easy. However, the lever cannot be removed from the main body 21 when the spindle member 81 is inserted by and engaged with the bearing portion 75. In short, while the lever 30 can be easily fitted to and removed from the main body 21, it is effectively prevented from inadvertently coming off from the main body 21.

FIG. 2 shows that the embodiment of breast pump 20 having the above described configuration in an unused or mint state. It is operated in a manner as described below.

Referring to FIG. 2, the lever 30 is fitted to the main body 21 and the bottle 41 is fitted to the main body 21 by way of the attach and detach portion 25.

Under this condition, the sleeve 32 of the lever 30 has been moved in the direction of arrow A2 by the leaf spring 33 so that the fixed piston 23 is located deep in the inside of the sleeve 32.

Then, the user applies the suction portion 22 to the breast by way of the opening thereof so that the breast may be surrounded by the suction portion 22. Then, the user applies the thumb of one of his or her hands to the bottle 41 and all or some of the fingers of the hand to the lever main body 31 of the lever 30 (not shown). Under this condition, the user closes the hand to operate the lever main body 31. Then, the lever 30 turns around the spindle in the direction of arrow A1 and also in the direction of arrow B1 as shown in FIG. 1 against the urging force of the leaf spring 33. During this process, the fixed piston 23 is moved relatively in the direction of arrow D to increase the volume of the internal space S1 of the sleeve 32. As a result, the negative pressure in the inside of the internal space S1 of the sleeve 32 is increased and the increased negative pressure is made to propagate to the suction portion vent passage 22a by way of the vent passage 27 and the cell 26a. Thus, negative pressure prevails in the inside of the suction portion 22 to suck mother's milk from the breast and the sucked mother's milk is driven to pass through the suction portion vent passage 22a and stored in the cell 26a. At this time, by convex portion 28a of the buffer 28 arranged in the inside of the suction portion 22, the breast drawn by the negative pressure in the inside of the suction portion 22 so as to abut, is pressed and provided an effect similar to that of massage.

At this time, since negative pressure prevails in the inside of the cell 26a, the oppositely disposed lateral walls 26b, 26c of the cellular valve 26 are drawn upward to close the slit 26d.

Then, as the user grasps the lever main body 31 in the direction of arrow BB until it comes to abut the outer edge of the bottle 41, the fixed piston 23 is moved relatively in the direction of arrow D. Eventually, the fixed piston 23 comes to the end of the relative movement and air infiltrates into the internal space S1 of the sleeve 32 through the leak portion 38. As a result, the negative pressure in the internal space S1 is quickly reduced and the internal pressure of the cell 26a that is held in communication with the internal space S1 by way of the vent passage 27 also comes close to the atmospheric pressure to decrease the effect of drawing the oppositely disposed lateral walls 26b, 26c of the cellular valve 26 upward. Then, the slit 26d constructed at the lower ends of the lateral walls 26b, 26c is opened by the weight of the mother's milk stored in the cell 26a and the milk stored in the cell 26a falls into the inside portion S3 of the bottle 41.

As a result of this process, the sucking pressure of the suction portion 22 applied to the breast of user falls.

Then, the lever 30 is returned to the position shown in FIG. 2 by the urging force of the leaf spring 33. Thus, operation of sucking milk can be continued by the user repeating the above process.

Therefore, with the embodiment of breast pump 20 according to the invention, the user is not required to handle the breast pump with his or her two hands unlike the conventional breast pump described above by referring to FIG. 3. So, the user only needs to use his or her dominant hand to handle the breast pump 20. Thus, the operation is simple and easy.

Additionally, the embodiment of breast pump 20 does not involve the use of a conventional long air pipe and the like, which is cumbersome and difficult to handle. So, the embodiment of breast pump 20 is remarkably superior in storage and portability.

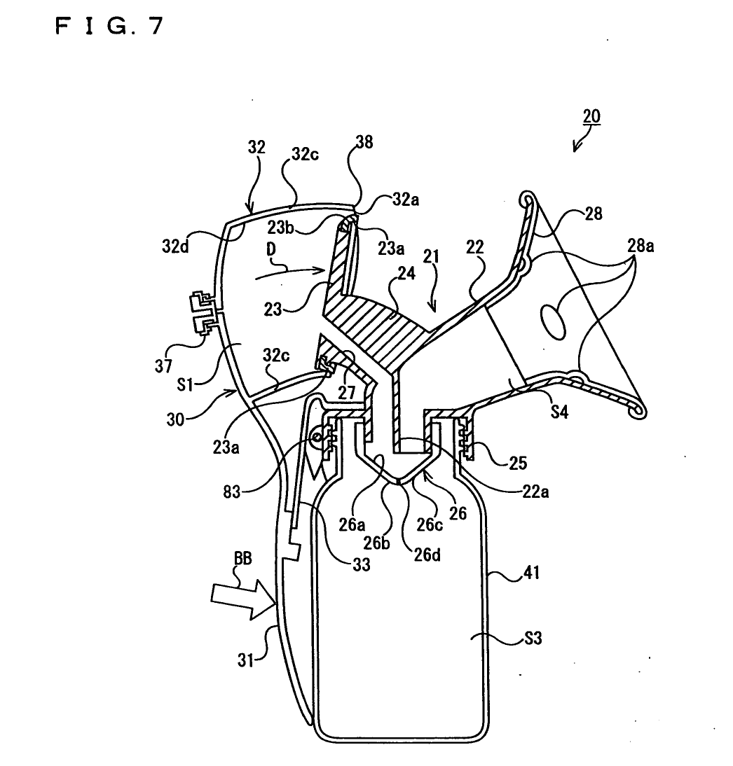

As described above and illustrated in FIGS. 2, 6 and 7, once the lever 30 is fitted to the main body 21, engagement of spindle holding structure of the lever 30 with the main body 21 is not detached any changeful position in the state that the bottle 41 is fitted to the main body 21. In other words, once the lever 30 is fitted to the main body 21, the status that main body 21 is fitted to the bottle 41, the situation is that the user can proceed under the operation of sucking milk, the lever is constructed not to come off from the main body 21.

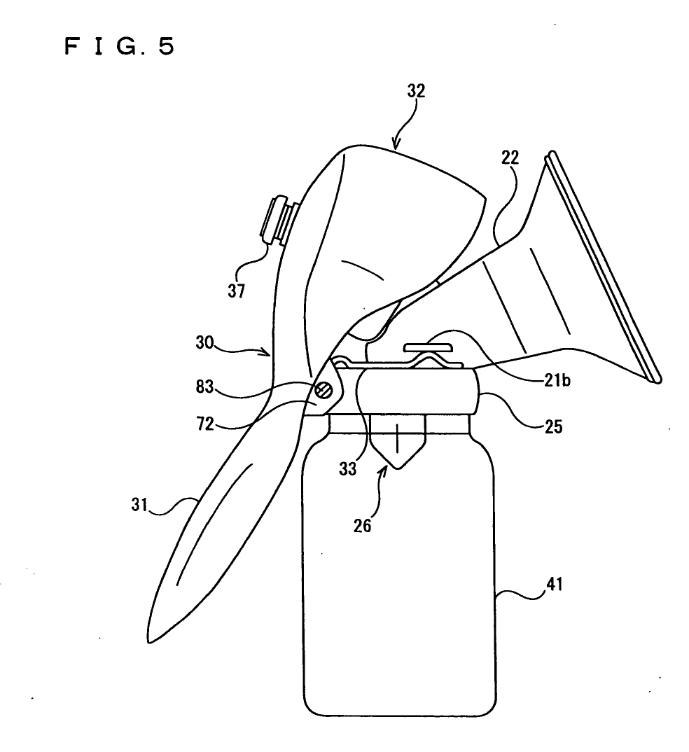

Thus, the lever 30 is securely held to the main body 21 only by the spindle member 81 be inserted into the spindle hole 76 and by operation for mutual engagement. Therefore, the assembling is very simple and easy. Additionally, the lever 30 can be removed simply by removing the spindle member 81 from the spindle hole 76 so that the assembling and disassembling is easy. Furthermore, in the breast pump 20, the leaf spring 33 as urging means is held at an end thereof by the holding means 21b of the main body 21 as shown in FIG. 5 and made at the other end thereof to abut the spring bearing 34 arranged near the front end of the lever main body 31 of the lever 30 in a manner as described above. Therefore, when the lever 30 is removed from the main body 21, it can be taken out simultaneously with ease and cleaned. When the urging means is a leaf spring 33 that is made of metal, stainless steel (SUS304) in particular, it is superior in durability and can surely rebound. Additionally, the leaf spring 33 can be taken out to sterilize the remaining components of the breast pump 20 by means of a chemical such as sodium hypophosphite and the like. In this case, this operation is conducted safely because the leaf spring 33 cannot be taken out without removing the lever 30 from the main body 21.

Furthermore, in the breast pump 20, the sleeve 32 is integrally formed at an end of the lever 30. Thus, the opening for inserting the fixed piston 23 and the sleeve 32 of which internal space are exposed can be cleaned by only removing the lever 30, so, the space for generating negative pressure has a simple configuration and can be cleaned with ease. Additionally, once the lever 30 is removed, the fixed piston 23 from which the sleeve 32 has been moved away comes to be exposed on the main body 21 so that it can also be cleaned with ease. In this case, since the sleeve 32 is made movable while the piston is fixed, the piston rod 24 of the fixed piston 23 does not require any junction such as link. So, the piston rod 24 is free from parts where dirt of dregs and the like of mother's milk and the like would easily adhere and is difficult to remove so that it can be cleaned with ease and held in a highly hygienic condition.

In short, the mechanism for generating negative pressure can be exposed for cleaning by a simple operation of removing the lever 30 from the main body 21.

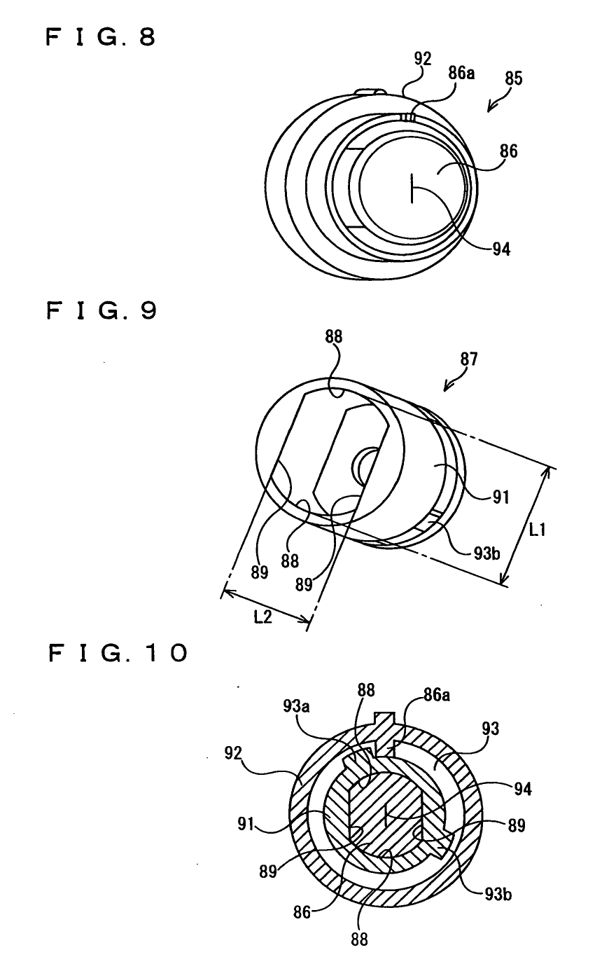

FIGS. 8 through 12 illustrate the valve structure of the pressure regulating means 37 arranged on the outer surface of the sleeve 32 of the breast pump 20.

FIG. 8 shows the valve main body 85 that is part of the pressure regulating means 37 and FIG. 9 shows the valve box portion 87 that is to be combined with the valve main body 85.

The valve box 91 of the valve box portion 87 is a bottomed cylinder having two pairs of oppositely disposed inner walls 88, 88 and 89, 89 in the inside. The pair of oppositely disposed inner walls 88, 88 are separated from each other by distance L1, which is greater than distance L2 by which the other pair of oppositely disposed inner walls 89, 89 are separated from each other. L1 is substantially equal to or slightly greater than the diameter of the circular valve body 86. The distance L2 is smaller than the diameter of the valve body 86.

As shown in FIG. 8, the valve main body 85 that is housed in the valve box 91 has a valve body 86 at the front end of a substantially flat and cylindrical base 92 that is made of a hard material. The valve body 86 has a circular outer periphery and a slit 94 located at the center thereof. At least a part of the valve body 86 where the slit 94 is formed is made of an elastic material such as rubber.

As for the situation that, the valve body 86 is housed in the valve case 91 shown in FIG. 9. FIG. 10 is a cross sectional view of the valve body 86 and the abstract of situation of a stopper.

Referring to FIG. 10, the stopper 86a projecting inward is arranged on the inner periphery of the base section 92. On the other hand, the valve case 91 is provided at a base section thereof with a guide groove 93 for receiving the stopper 86a. The guide groove 93 contains therein a first positioning portion 93a projecting outward and a second positioning section 93b spaced from the first positioning section 93a by ¼ of the circumference or more.

As seen from FIG. 10, when the stopper 86a abuts the first positioning section 93a, the valve main body 86 is housed in the valve case 91 in such a way that the slit 94 locates so as to connect the inner walls 88, 88. So, the longitudinal direction of the slit 94 agrees with that of the longer distance L1 so that the slit 94 is closed. Additionally, since the distance L2 is small relative to the disk-shaped valve body 86, the slit 94 is pushed and pressed to the direction to be pushed. Thus, the pressure regulating means 37 is closed and the inner space S1 is isolated from the atmosphere.

On the other hand, as the valve main body 86 is turned in the valve case 91 and the base portion 93 is relatively turned in the direction indicated by an arrow, the distance that contains the longitudinal direction of the slit 94 is gradually reduced and the opposite ends 94a, 94a of the slit 94 which was crushed the slit becomes open to allow air to pass through it slightly. As a result, the negative pressure in the internal space S1 of the sleeve 32 is slightly reduced.

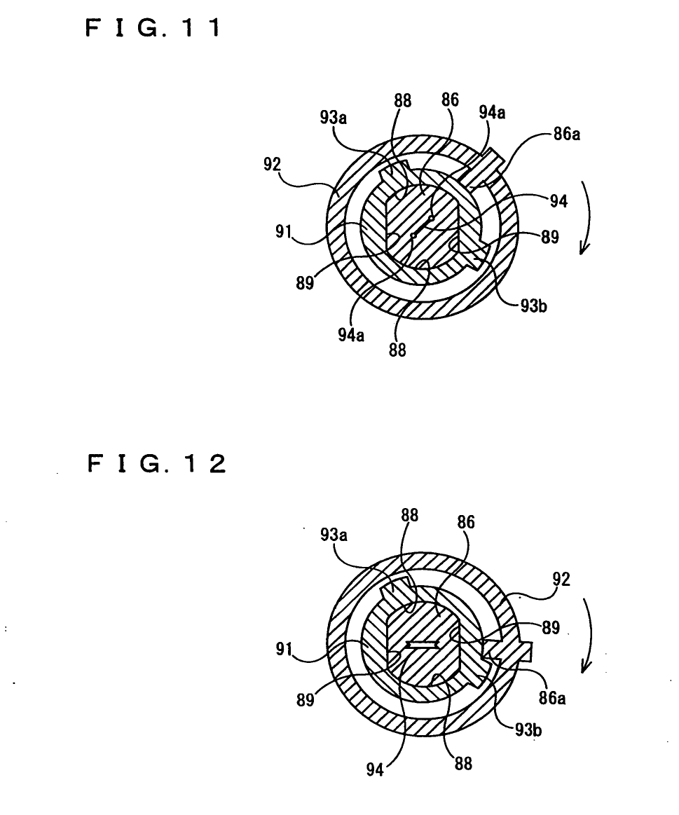

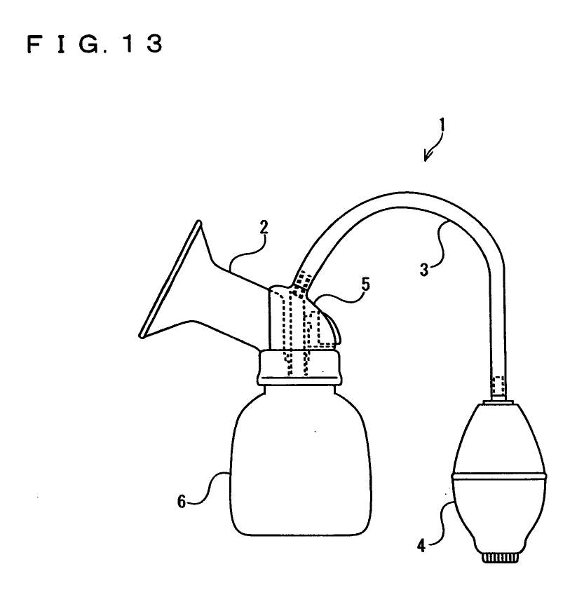

Then, as shown in FIG. 11, as the valve main body 86 is turned further in the valve box 91 in the direction indicated by the arrow until the stopper 86a abuts the second positioning portion 93b, the distance that contains longitudinal direction of the slit 94 is further reduced. Since the longitudinal direction of the slit 94 is in the direction of the shorter distance L2, the slit 94 becomes filly open. As a result, air passes at an enhanced rate to remarkably reduce the negative pressure in the internal space S1 of sleeve 32.

By the way, the present invention is by no means limited to the above described embodiment.

For instance, the outer surface of the lever main body of the lever 30 may be provided with anti-slippery undulations or a finger hook so that the lever 30 may be handled with ease.

After the above described embodiment, or example of modification some of the components may be omitted if needed or combined with other components which is not explained herein.

### INDUSTRIAL APPLICABILITY

As described above in detail, the present invention provides a manual breast pump that can be conveniently operated by hand.

1. A manual breast pump comprising:
a breast pump main body having a substantially conical suction portion to be applied to a breast, and an attachable/detachable portion detachably fitted to a bottle to communicate with the bottle;
a lever that is elongated in a first direction and is detachably supported by said breast pump main body at a middle part thereof by a shaft portion, said lever being rotatable about the shaft portion, wherein
one of said lever and main body define a predetermined internal space that contains a fixed piston, and
said lever is in communication with the predetermined internal space and is configured to cause milk to be sucked from said suction portion by generating negative pressure in said internal space due to change of the internal space that contains the fixed piston by relative reciprocating motion of the piston which is caused by movement of an end of said lever; and
a negative pressure structure including, a vent passage communicating with said suction portion, the fixed piston located at an end of the vent passage, wherein a sleeve is integrally located at an end of said lever such that the piston of said breast pump main body is insertable in the sleeve so as to form a piston space that communicates with said vent passage, said piston configured to slide in an inside area of said sleeve when said lever is rotated so as to generate negative pressure by rotating the lever in the piston space defined by an inner surface of said sleeve and said fixed piston.

2. The breast pump recited in claim 1, wherein the shaft portion is rotative and supportive and the lever has a bearing portion located at a side of said lever and the breast pump main body has a bearing portion located at a side of said breast pump main body,
the shaft portion includes a shaft member that is insertable into spindle holes located in the bearing portions, and the shaft member includes an engaging structure configured to engage a portion of said bearing portions.

3. The breast pump recited in claim 1, wherein at least an inner wall of said sleeve has a curved profile that corresponds to a locus of rotary motion of an end of the lever.

4. The breast pump recited in claim 1, wherein an outer periphery of the piston is elastic and inclined toward the lever.

5. The breast pump recited in claim 1, wherein the piston is not in airtight contact with an inner wall of the sleeve when the lever is in a predetermined position.

6. The breast pump recited in claim 1, further comprising:
a pressure regulating device located on an outer surface of a closed end of the sleeve.

7. The breast pump recited in claim 6, wherein the pressure regulating device includes,
a valve box having at least two pairs of oppositely disposed inner walls, with a first distance that separates one of the pairs of oppositely disposed inner walls being greater than a second distance that separates the other pair of oppositely disposed inner walls, and
a valve body located in the valve box and made of an elastic material, the valve body having a substantially circular outer periphery and a slit for allowing air to pass through, and the opening of said slit being adjustable by rotating the valve body in said valve box.

8. The breast pump recited in claim 2, wherein at least an inner wall of said sleeve has a curved profile that corresponds to a locus of rotary motion of an end of the lever.

9. The breast pump recited in claim 2, wherein an outer periphery of the piston is elastic and inclined toward the lever.

10. The breast pump recited in claim 2, wherein the piston is not in airtight contact with an inner wall of the sleeve when the lever is in a predetermined position.

11. The breast pump recited in claim 2, further comprising:
a pressure regulating device located on an outer surface of a closed end of the sleeve.

12. The breast pump recited in claim 5, further comprising:
a pressure regulating device located on an outer surface of a closed end of the sleeve.

13. A manual breast pump comprising:
a breast pump main body having a substantially conical suction portion to be applied to a breast, and an attachable/detachable portion detachably fittable to a bottle;
a lever that is elongated in a first direction and is located adjacent to said breast pump main body, said lever being rotatable about a shaft portion, wherein
one of said lever and main body define a predetermined internal space in which a piston is located, and
said lever is in communication with the predetermined internal space and is configured to cause milk to be sucked from said suction portion by generating negative pressure in said internal space due to change of the internal space by relative reciprocating motion of the piston which is caused by a movement of said lever; and
a negative pressure structure including,
a vent passage communicating with said suction portion, the piston located adjacent the vent passage, wherein said lever includes a sleeve in which the piston is insertable to form a piston space defined by an inner surface of said sleeve and said piston, the piston space communicating with said vent passage, said piston being configured to slide in said sleeve when said lever is moved so as to generate negative pressure by moving the lever.

14. The breast pump as recited in claim 13, wherein the lever has a bearing portion arranged at a side of said lever and the breast pump main body has a bearing portion arranged at a side of said breast pump main body,
the shaft portion includes a shaft member that is insertable into spindle holes located in the bearing portions, and the shaft member includes an engaging structure configured to engage a portion of said bearing portions.

15. The breast pump recited in claim 13, further comprising:
a bottle connected to the main body by the attachable/detachable portion.

16. The breast pump recited in claim 13, wherein the sleeve is integral with the end of the lever.

17. The breast pump recited in claim 13, wherein the piston is a fixed piston relative to the main body.

18. The breast pump recited in claim 14, wherein the lever bearing portion and the pump main body bearing portion are laid one on the other.

19. The breast pump recited in claim 13, wherein the sleeve is configured such that when the lever is in a predetermined position, the internal space is open to atmosphere.

20. The breast pump recited in claim 13, further comprising:
a pressure regulating device located on the sleeve,
wherein the pressure regulating device includes a valve box having at least two pairs of oppositely disposed inner walls, with a first distance that separates one of the pairs of oppositely disposed inner walls being greater than a second distance that separates the other pair of oppositely disposed inner walls, and
a valve body located in the valve box and made of an elastic material, the valve body having a substantially circular outer periphery and a slit for allowing air to pass through, and the opening of said slit being adjustable by rotating the valve body in said valve box.
